(19) United States
(12) Patent Application Publication
(10) Pub. No.: US 2018/0161216 A1
(43) Pub. Date: Jun. 14, 2018

(54) BIKINI SATIN INCONTINENCE WEAR WITH PERFUME POCKET

(71) Applicant: Cynthia Herchert, Prescott, AZ (US)

(72) Inventor: Cynthia Herchert, Prescott, AZ (US)

(21) Appl. No.: 15/835,429

(22) Filed: Dec. 7, 2017

Related U.S. Application Data
(60) Provisional application No. 62/431,761, filed on Dec. 8, 2016.

(51) Int. Cl. A61F 13/496; A61F 13/62; A61F 13/51; A61F 13/491; A61F 13/53

(52) U.S. Cl. CPC A61F 13/496; A61F 13/622; A61F 2013/5109; A61F 13/491; A61F 13/53; A61F 13/51

(57) ABSTRACT

An improved underwear garment includes an outer layer; an inner layer; a waist band; two leg openings; a pocket member; and at least one scented member. The inner layer is adapted to absorb any liquid discharge from a user and deter the liquid discharge from reaching the outer layer. The waist band is attached to an upper edge of the outer layer. The pocket member is attached to an outer surface of the outer layer and forms an interior volume. The at least one scented member is removably located within the interior volume of the pocket member and is adapted such that the at least one scented member can provide a scent to the improved underwear garment while the user is wearing the improved underwear garment, and wherein when the at least one scented member no longer provides a scent it can be removed and replaced with another scented member.

### CROSS-REFERENCE TO RELATED APPLICATION

The present application is related to and claims priority from prior provisional application Ser. No. 62/431,761, filed Dec. 8, 2016 which application is incorporated herein by reference.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

The following includes information that may be useful in understanding the present invention(s). It is not an admission that any of the information provided herein is prior art, or material, to the presently described or claimed inventions, or that any publication or document that is specifically or implicitly referenced is prior art.

### 1. FIELD OF THE INVENTION

The present invention relates generally to the field of incontinence undergarments and more specifically relates to an improved underwear garment in the form of a flattering bikini, which are specially configured for urine absorbency including a pocketed time-release, scented tablet or scented cotton strips, structured and arranged to provide a simple and sanitary means of preventing leaks and odor due to incontinence, and to enable the wearer to remain stylish while maintaining a level of freshness and comfort throughout the day.

### 2. DESCRIPTION OF THE RELATED ART

Incontinence affects 25 million Americans and is a public health problem of enormous proportions, resulting in an annual cost of treatment that exceeds 16 billion. This condition is one of the most common reasons why people enter nursing homes, where weary caregivers crumble under the strain of constantly changing diapers and cleaning wet sheets. There are, of course, varying degrees of incontinence. Some people have occasional leaks when they cough, sneeze, or exercise. Others, however, have little or no control of their urine, and this affects virtually every aspect of their existence. While incontinence mostly affects older adults, it is not confined to them. It is seen in people of all ages, although the incidence rises with each passing decade. Although it is widely known that women are twice as likely to develop the condition as men, a significant percentage of the male population have to deal with incontinence on a daily basis.

While a physical ailment, perhaps one of the most distressing side effects of incontinence is psychological. Particularly, the lack of urinary control invariably results in the staining of undergarments and outer clothing, which is very embarrassing and demeaning to sufferers. In addition to humiliation, incontinence can also ruin garments, resulting in costly dry cleaning charges and even clothing replacement. To help protect their clothing, as well as their self-confidence, patients with these conditions turn to absorbent, disposable diaper-like garments to serve as a shield between their clothing and the source of any seepage. While adult diapers can be very effective in achieving their intended purpose, these units tend to be extremely bulky and uncomfortable to wear.

Various attempts have been made to solve problems found in incontinence undergarments art. Among these are found in: U.S. Pat. No. 5,593,398 to Chester L. Weimer; U.S. Publication No. 2004/0127866 to Thomas Odorzynski; and U.S. Publication No. 2012/0123376 to Maria Alves. This prior art is representative of incontinence undergarments.

Ideally, an improved underwear garment should be user-friendly and safe in-use and, yet should operate reliably and be manufactured at a modest expense. Thus, a need exists for improved underwear garment in the form of a flattering bikini, which are specially configured for urine absorbency including a pocketed time-release, scented tablet or scented cotton strips, structured and arranged to provide a simple and sanitary means of preventing leaks and odor due to incontinence, and to enable the wearer to remain stylish while maintaining a level of freshness and comfort throughout the day and to avoid the above-mentioned problems.

### BRIEF SUMMARY OF THE INVENTION

In view of the foregoing disadvantages inherent in the known incontinence undergarments art, the present invention provides an Improved Underwear Garment (Entitled Bikini Satin Incontinence Wear with Perfume Pocket). The general purpose of the present invention, which will be described subsequently in greater detail is to provide an improved underwear garment in the form of a flattering bikini, which are specially configured for urine absorbency including a pocketed time-release, scented tablet or scented cotton strips, structured and arranged to provide a simple and sanitary means of preventing leaks and odor due to incontinence, and to enable the wearer to remain stylish while maintaining a level of freshness and comfort throughout the day.

An improved underwear garment comprising: an outer layer; an inner layer; a waist band; two leg openings; a pocket member; and at least one scented member. The outer layer is formed from a flexible material. The inner layer is attached to an inner surface of said outer layer and is formed from an absorbent material. The inner layer is adapted to absorb any liquid discharge from a user and deter the liquid discharge from reaching the outer layer. The waist band is formed of a stretchable material. The waist band is attached to an upper edge of the outer layer. The two leg openings are spaced and from one another and located at a lower section of the outer layer. The pocket member is attached to an outer surface of the outer layer and forms an interior volume.

The at least one scented member is removably located within the interior volume of the pocket member and is adapted such that the at least one scented member can provide a scent to the improved underwear garment while the user is wearing the improved underwear garment, and wherein when the at least one scented member no longer provides a scent it can be removed and replaced with another scented member.

The present invention holds significant improvements and serves as an Improved Underwear Garment. For purposes of summarizing the invention, certain aspects, advantages, and novel features of the invention have been described herein. It is to be understood that not necessarily all such advantages may be achieved in accordance with any one particular embodiment of the invention. Thus, the invention may be embodied or carried out in a manner that achieves or optimizes one advantage or group of advantages as taught herein without necessarily achieving other advantages as may be taught or suggested herein. The features of the invention which are believed to be novel are particularly pointed out and distinctly claimed in the concluding portion of the specification. These and other features, aspects, and advantages of the present invention will become better understood with reference to the following drawings and detailed description.

### BRIEF DESCRIPTION OF THE DRAWINGS

The figures which accompany the written portion of this specification illustrate embodiments and method(s) of use for the present invention, Improved Underwear Garment (Entitled Bikini Satin Incontinence Wear with Perfume Pocket), constructed and operative according to the teachings of the present invention.

FIG. 1 shows a perspective view illustrating an improved underwear garment according to an embodiment of the present invention.

FIG. 2A is a perspective view illustrating a pocket member having a flap portion including a removable fastening having hook and loop material including at least one scented member in the form of a time released scented capsule member of the improved underwear garment according to an embodiment of the present invention.

FIG. 2B is a perspective view illustrating a pocket member having a flap portion including a snap fastener including at least one scented member in the form of a scented pill member of the improved underwear garment according to an embodiment of the present invention.

FIG. 2C is a perspective view illustrating a pocket member having a flap portion including at least one scented member in the form of a scented strip member of the improved underwear garment according to an embodiment of the present invention.

FIG. 3 is a perspective view illustrating the improved underwear garment in an in-use condition according to an embodiment of the present invention of FIG. 1.

The various embodiments of the present invention will hereinafter be described in conjunction with the appended drawings, wherein like designations denote like elements.

### DETAILED DESCRIPTION

As discussed above, embodiments of the present invention relate to an incontinence undergarment and more specifically relates to an improved underwear garment (entitled Bikini Satin Incontinence Wear with Perfume Pocket) in the form of a flattering bikini, which are specially configured for urine absorbency including a pocketed time-release, scented tablet or scented cotton strips, structured and arranged to provide a simple and sanitary means of preventing leaks and odor due to incontinence, and to enable the wearer to remain stylish while maintaining a level of freshness and comfort throughout the day.

Generally speaking, the Bikini Satin Incontinence Wear with Perfume Pocket comprising a novel product offering consumers a practical solution to the aforementioned challenges. As the name implies, the Bikini Satin Incontinence Wear with Perfume Pocket comprises a specially designed line of wearable absorbent garments, configured in a unisex bikini design that is appropriate for the female genitalia as well as male, that is specially designed for urination absorption. Further, as the name of the product implies, these accessories would be enhanced with a scented tablet to further increase freshness while the Bikini Silk Depends are worn.

Worn below the belly button, and offered in a variety of sizes to accommodate users of any stature, the Bikini Satin Incontinence Wear with Perfume Pocket would be presented as an undergarment fashioned from sleek silky material, with the interior of each garment comprised of a copious, heavy duty, yet non-cumbersome, bio-degradable, absorbent padding. Thus constructed, the Bikini Satin Incontinence Wear with Perfume Pocket would be able to collect a significant amount of discharge, and keep it confined within the layers without any liquid seeping through to the outer material.

The front of the lower portion of the Bikini would boast a Velcro-enhanced pouch or pocket, into which the unit's perfume tablet or scented cotton strips would be placed. These could be offered in a time-release capsule or material that dissolves as the units are worn, infusing the garment and the wearer with a fresh, pleasant scent all day long. All undergarments in the Bikini Satin Incontinence Wear with Perfume Pocket line would be durably sealed on all sides to ensure against leakage and discomfort to the wearer. In addition, sizes, styles, and fragrances would be produced for both women and men. The Bikini Satin Incontinence Wear with Perfume Pocket would provide consumers who are afflicted with incontinence a simple and effective means of wetness and odor protection.

An aesthetically pleasing, wearable bikini unit with various styles that are specifically configured to fit the male and female genitalia, this practical unit would offer consumers a comfortable, secure, and fashionable fit. In this manner, women and men who suffer from incontinence and similar conditions would be able to go about their daily routines with confidence, assured that the Bikini Satin Incontinence Wear with Perfume Pocket will allow them to avoid the embarrassment of walking around in wet or visibly stained clothing. In addition to preventing accidental humiliation, the Bikini Satin Incontinence Wear with Perfume Pocket would provide an immediate infusion of time released fresh scent, so that throughout the day, and in the event of an accident, any odor is conveniently masked by the perfume tablet or scented cotton strips.

Comfortably worn, the Bikini Satin Incontinence Wear with Perfume Pocket would eliminate the need to wear bulky, uncomfortable standard adult diapers, which can be an eye sore and an embarrassment under fitted clothing. Secure and comfortable, this clever product would offer a discreet, effective, and easy solution for incontinence and similar problems. As such, the Bikini Satin Incontinence Wear with Perfume Pocket line would readily enhance the daily lives of millions of women and men.

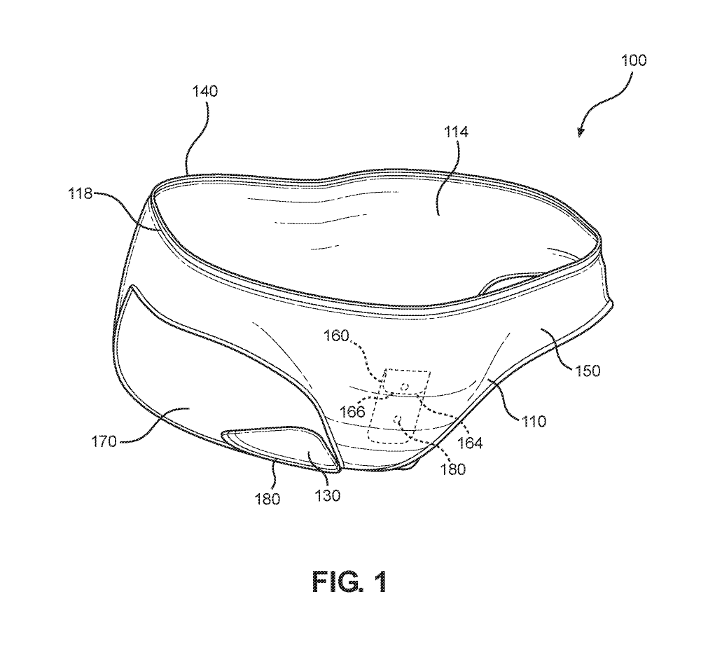

Referring now to FIGS. 1-3, showing perspective views illustrating improved underwear garment 100 according to an embodiment of the present invention of FIG. 1.

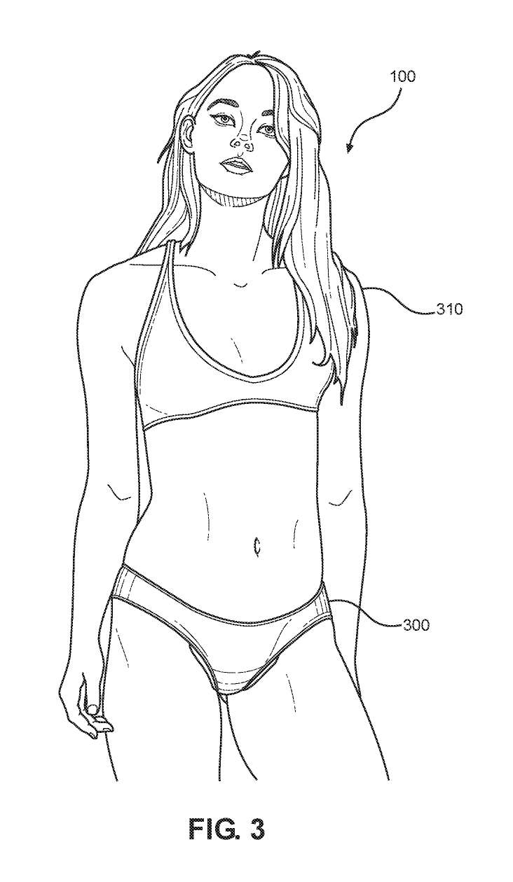

Improved underwear garment 100 comprising: outer layer 110; inner layer 130; waist band 140; two leg openings 150; pocket member 160; and at least one scented member 180. Outer layer 110 is formed from a flexible material. Outer layer 110 is formed from silk. Inner layer 130 is attached to inner surface 114 of outer layer 110 and is formed from an absorbent material. Inner layer 130 is adapted to absorb any liquid discharge from user 310 and deter the liquid discharge from reaching outer layer 110 as shown in in-use condition 300 of FIG. 3. Wherein inner layer 130 is attached to and located upon a substantial portion of the inner surface of outer layer 110. Wherein inner layer 130 is formed from a bio-degradable material.

Waist band 140 is formed of a stretchable material. Waist band 140 is attached to upper edge 118 of outer layer 110. Two leg openings 150 are spaced and from one another and located at a lower section of outer layer 110. Two leg openings 150 are surrounded by stretchable material to thereby allow leg opening 150 to vary in circumference. Wherein waist band 140 and two leg openings 150 are adapted such that when in use improved underwear garment 100 forms a seal between improved underwear garment 100 and user 310 to ensure against any leakage of any liquid discharge from improved underwear garment 100.

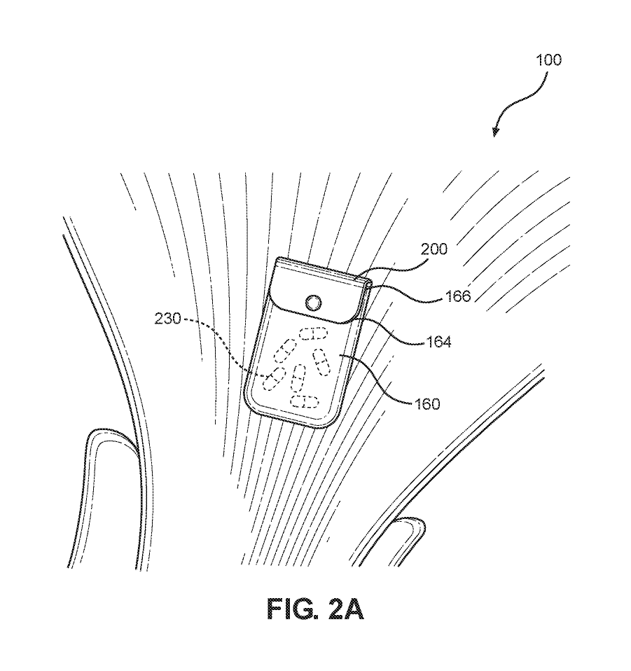

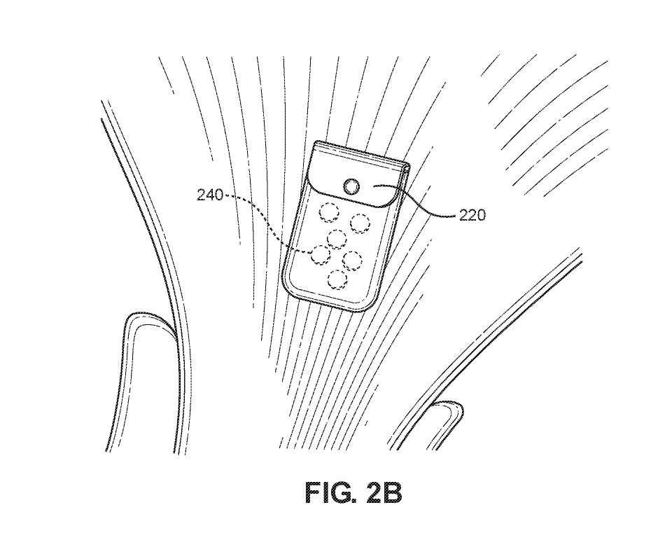

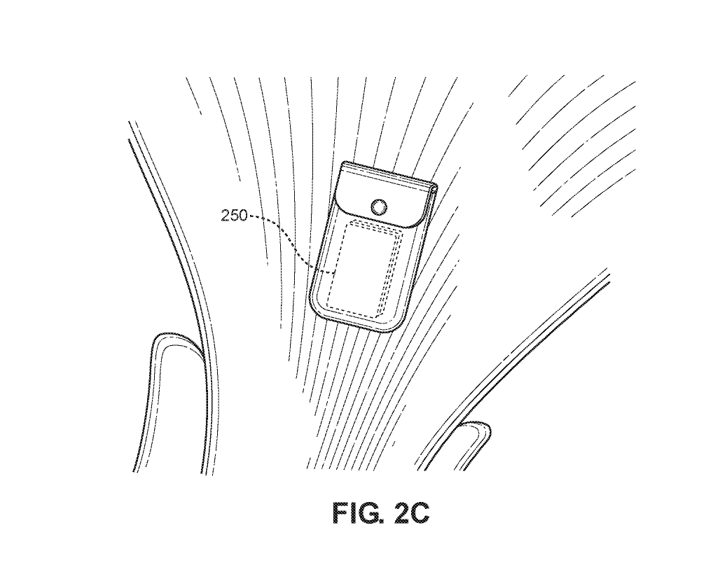

Pocket member 160 is attached to an outer surface of outer layer 110 and forms an interior volume 170. Wherein pocket member 160 includes flap portion 164 adapted to releasably enclose interior volume 170; and wherein flap portion 164 is removably attached to an outer surface of pocket member 160 via removable fastener 166. Removable fastener 166 is formed from hook and loop material 200 as shown in FIG. 2A. Removable fastener 166 is formed as snap fastener 220 as shown in FIG. 2B.

At least one scented member 180 is removably located within interior volume 170 of pocket member 160 and is adapted such that at least one scented member 180 can provide a scent to the improved underwear garment 100 while user 310 is wearing improved underwear garment 100, and wherein when at least one scented member 180 no longer provides a scent it can be removed and replaced with another scented member 180. Wherein at least one scented member 180 is time released scented capsule member 230 as shown in FIG. 2A. Wherein said at least one scented member 180 is adapted to dissolve over time. Wherein at least one scented member 180 is formed as scented pill member 240 as shown in FIG. 2B. Wherein at least one scented member 180 is formed as scented strip member 250 as shown in FIG. 2C. Wherein scented strip member 250 is formed from cotton. Wherein the cotton scented strip member 250 includes perfume thereon.

The embodiments of the invention described herein are exemplary and numerous modifications, variations and rearrangements can be readily envisioned to achieve substantially equivalent results, all of which are intended to be embraced within the spirit and scope of the invention. Further, the purpose of the foregoing abstract is to enable the U.S. Patent and Trademark Office and the public generally, and especially the scientist, engineers and practitioners in the art who are not familiar with patent or legal terms or phraseology, to determine quickly from a cursory inspection the nature and essence of the technical disclosure of the application.

What is claimed is:

1. An improved underwear garment comprising:
an outer layer;
wherein said outer layer is formed from a flexible material;
an inner layer;
wherein said inner layer is attached to an inner surface of said outer
layer and is formed from an absorbent material; wherein said inner layer is adapted to absorb any liquid discharge from a user and deter said liquid discharge from reaching said outer layer;
a waist band;
wherein said waist band is formed of a stretchable material; and
wherein said waist band is attached to an upper edge of said outer layer;
two leg openings;
wherein said two leg openings are spaced and from one another and located at a lower section of said outer layer;
a pocket member;
wherein said pocket member is attached to an outer surface of said outer layer and forms an interior volume; and
at least one scented member;
wherein said at least one scented member is removably located within said interior volume of said pocket member and is adapted such that said at least one scented member can provide a scent to said improved underwear garment while said user is wearing said improved underwear garment, and wherein when said at least one scented member no longer provides a scent it can be removed and replaced with another scented member.

2. The improved underwear garment of claim 1, wherein said two leg openings are surrounded by stretchable material to thereby allow said leg opening to vary in circumference.

3. The improved underwear garment of claim 1, wherein said inner layer is attached to and located upon a substantial portion of the inner surface of said outer layer.

4. The improved underwear garment of claim 1, wherein said at least one scented member is a time released scented capsule member.

5. The improved underwear garment of claim 1, wherein said pocket member includes a flap portion adapted to releasably enclose said interior volume; and wherein said flap portion is removably attached to an outer surface of said pocket member via a removable fastener.

6. The improved underwear garment of claim 5, wherein said removable fastener is formed from hook and loop material.

7. The improved underwear garment of claim 5, wherein said removable fastener is formed as a snap fastener.

8. The improved underwear garment of claim 1, wherein at least one scented member is formed as a scented pill member.

9. The improved underwear garment of claim 1, wherein at least one scented member is formed as a scented strip member.

10. The improved underwear garment of claim 9, wherein said scented strip member is formed from cotton.

11. The improved underwear garment of claim 1, wherein said cotton scented strip member includes perfume thereon.

12. The improved underwear garment of claim 1, wherein said outer layer is formed from silk.

13. The improved underwear garment of claim 1, wherein said inner layer is formed from a bio-degradable material.

14. The improved underwear garment of claim 1, wherein said at least one scented member is adapted to dissolve over time.

15. The improved underwear garment of claim 2, wherein said waist band and said two leg openings are adapted such that when in use said improved underwear garment forms a seal between said improved underwear garment and said user to ensure against any leakage of any liquid discharge from said improved underwear garment.
